(19) United States
(12) Patent Application Publication
(10) Pub. No.: US 2008/0228172 A1
(43) Pub. Date: Sep. 18, 2008

(54) INTRAVENOUS ACCESS SAFETY DEVICE

(76) Inventor: Thomas S. Petry, Lafayette, IN (US)

Correspondence Address: BINGHAM MCHALE LLP, 2700 MARKET TOWER, 10 WEST MARKET STREET, INDIANAPOLIS, IN, 46204-4900, US

(21) Appl. No.: 12/035,103

(22) Filed: Feb. 21, 2008

Related U.S. Application Data
(60) Provisional application No. 60/914,330, filed on Apr. 27, 2007.
(60) Provisional application No. 60/894,950, filed on Mar. 15, 2007.

(51) Int. Cl. A61M 25/18

(52) U.S. Cl. 604/533

(57) ABSTRACT

An intravenous catheter system comprises a Y-shape, where one arm includes a needle for catheterization, another accommodates a syringe assembly, and the third is threaded for attachment to IV tubing. A syringe assembly is threadedly attached to the Y for cannulation, after which the needle is withdrawn into a tube and lugs prevent re-exposure of the needle's point. A basket connector/valve in the third arm substantially prevents flow through the third arm when no IV tubing is attached thereto. An adhesive strip with backing is wrapped around the Y, perpendicular to its axis, and adheres (or, alternatively, is integrally attached) in its middle to the Y. After cannulation, the backing is removed and the ends of the strip are folded to be parallel to the catheter where it attaches to the Y.

### REFERENCE TO RELATED APPLICATIONS

This application claims the benefit of U.S. Provisional Patent Applications 60/894,950, filed Mar. 15, 2007, and 60/914,330, filed Apr. 27, 2007, both with title INTRAVENOUS ACCESS SAFETY DEVICE, the disclosures of which are hereby incorporated by reference.

### FIELD

The present disclosure relates generally to an intravenous safety device, and more particularly relates to a device that provides intravenous access to a patient without the risk of needle stick and without blatant blood exposure.

### SUMMARY

Intravenous (“IV”) catheters are commonly used in medical settings to provide fluid replacement, nourishment, and a port for medication administration to a patient. Such catheters commonly consist of a hollow plastic tube that is inserted into the patient's vein with the assistance of a sharp, hollow, beveled needle that is inserted into the plastic IV catheter. In use, the healthcare worker locates the patient's vein, punctures the skin and cannulates the vein. The needle is then withdrawn from the catheter and an IV fluid line is connected to the catheter.

There are several difficulties with this procedure. First, when the needle is withdrawn from the catheter, blood flows freely out onto the patient's skin and onto the health worker's hands. Second, the needle itself must be disposed of to avoid the potential of a needle stick occurring. However, the healthcare worker is occupied with connecting the IV quickly to avoid unnecessary blood loss through the catheter and therefore often places the needle temporarily on the patient's bed or bedside table, where it can get lost or accidentally puncture either the patient or the healthcare worker. It is important to not only prevent blood or fluid leakage after the needle is withdrawn, but also to provide for safe needle encapsulation and ultimate disposal.

Preventing exposure to blood and blood products is critical because of the possibility that the healthcare worker may be exposed to diseases such as Hepatitis and H.I.V. Multiple devices have been developed in an attempt to minimize this risk. Most deal with the potential for needle sticks by providing various means of encapsulating the needle after IV access is obtained. Several provide either automatic needle withdrawal, such as U.S. Pat. No. 6,547,762, or semiautomatic devices such as U.S. Pat. No. 4,747,831. One disadvantage of these devices is the inability to readjust the catheter if it accidentally dislodges from inside the vein while the needle is being retracted, which is a common occurrence. In these prior art devices the venous catheter cannot be advanced into the vein until the needle is retracted, commonly resulting in a failed vein cannulation. This then requires removal of the catheter and initiating the entire procedure over again, including another unnecessary needle stick to the patient. Other manual devices for encapsulating the needle are often cumbersome or difficult to use, as they make identifying venous access difficult or are unwieldy to operate because of their size or design.

The problem of backflow of blood through the catheter has not been addressed as frequently. While some attempts have been made to prevent blood or fluid leakage after the needle is withdrawn, such attempts have not provided for safe needle encapsulation and ultimate disposal.

It would be desirable to have an IV catheter assembly that satisfies the need for easy medication delivery without disrupting the administration of the IV fluid delivery. Multiple “Y” adapters exist, such as U.S. Pat. No. 6,221,065, which can be attached to the IV catheter once it is inserted into the vein. However, these often unduly restrict the flow of fluid through them by their design, and do not allow for “wide open” administration of fluids as is often required in emergency situations.

It would be desirable to have a device that provides for safe encapsulation of the needle after venous access is obtained, and allows for readjustment of the needle and catheter if necessary to gain optimal cannulation prior to said encapsulation. It would also be desirable if such a device also prevented blatant blood exposure by preventing backflow of blood after the needle is removed from the catheter. It would be further desirable if such a device also provided dual access to the circulatory system in the form of a closed connection with IV fluids and a second port which allowed administration of medications without disrupting the flow of said IV fluids. It would also be desirable if the withdrawal of the needle from the catheter could be accomplished by the medical professional without the use of both hands.

### BRIEF DESCRIPTION OF THE DRAWINGS

FIG. 1 is a side perspective view of a first embodiment of the present technology.

FIG. 2 is an enlarged perspective view of the first embodiment, as shown in FIG. 1.

FIG. 3 is another enlarged perspective view of the first embodiment, as shown in FIG. 1.

FIG. 4 is an enlarged perspective view of the first embodiment, as shown in FIG. 1, showing the needle permeating both the syringe assembly membrane and the membrane of the syringe arm of the connector assembly.

FIG. 5 is a side view of an alternative embodiment that allows one-handed operation.

FIG. 6 is an enlarged perspective view of the alternative embodiment, as shown in FIG. 5.

FIG. 7 is an end view of the alternative embodiment, as viewed from the right side of FIG. 5.

FIG. 8 is an end view of the alternative embodiment, as shown in FIG. 5, showing the relationship between the hollow tube and outer sleeve in isolation.

FIG. 9 is a cross-sectional view of a syringe arm and adhesive bandage according to a second alternative embodiment.

FIG. 10 is a perspective view of a syringe arm and adhesive bandage according to the second alternative embodiment.

FIG. 11 is a plan view of a syringe and adhesive bandage according to the second alternative embodiment.

FIG. 12 is another plan view of a syringe and adhesive bandage as secured to a patient according to the second alternative embodiment.

### DESCRIPTION

For the purposes of promoting an understanding of the principles of the technology and presenting its currently understood best mode of operation, reference will now be made to the embodiments illustrated in the drawings and specific language will be used to describe the same. It will nevertheless be understood that no limitation of the scope of the disclosure or claims is thereby intended, with such alterations and further modifications in the illustrated device and such further applications of the principles of the technology as illustrated therein being contemplated as would normally occur to one skilled in the art to which the disclosure relates.

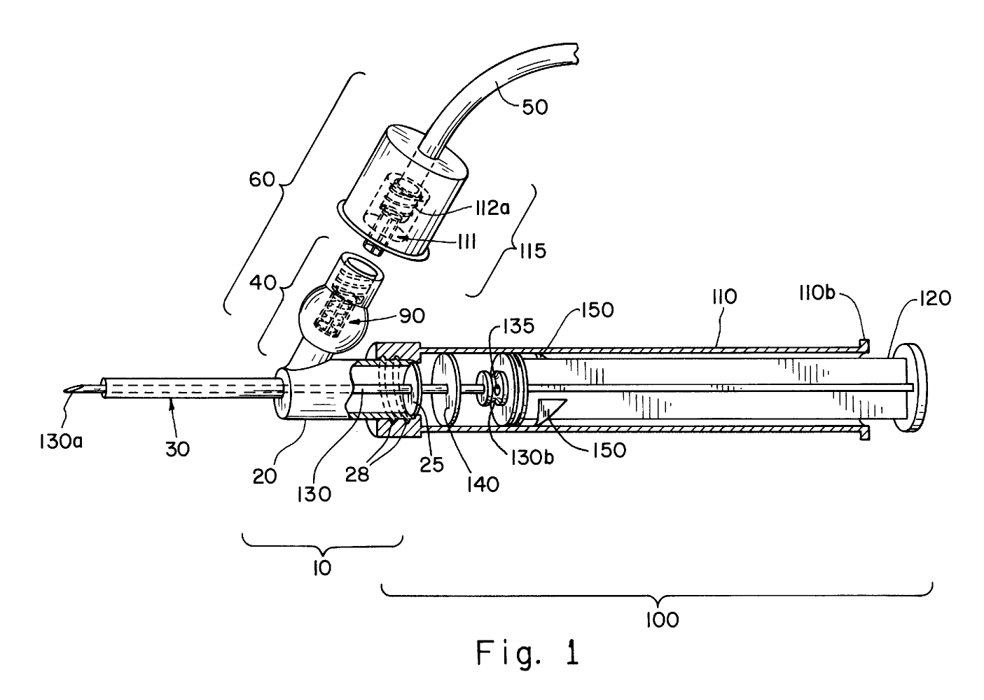

A first embodiment of the present technology presents a device that provides intravenous access to a patient without the risk of needle stick and without blatant blood exposure. As shown in FIGS. 1-4, the present disclosure includes a modified intravenous (“IV”) Y-junction 10 including a syringe arm portion 20, a catheter portion 30 arranged in opposing alignment with the syringe arm 20, a fluid-impermeable membrane 25 arranged internally of the syringe arm 20 opposite the catheter end 30, and an intravenous IV connector arm portion 40. The IV connector arm 40 is offset preferably at approximately 45 to 60 degrees from the syringe arm 20, though other embodiments have greater or lesser angles between the two arms. Syringe arm 20 is further provided with external threads 28 for mating with a syringe assembly 100. The Y-junction 10 could also be mated with syringe apparatuses currently available in the industry.

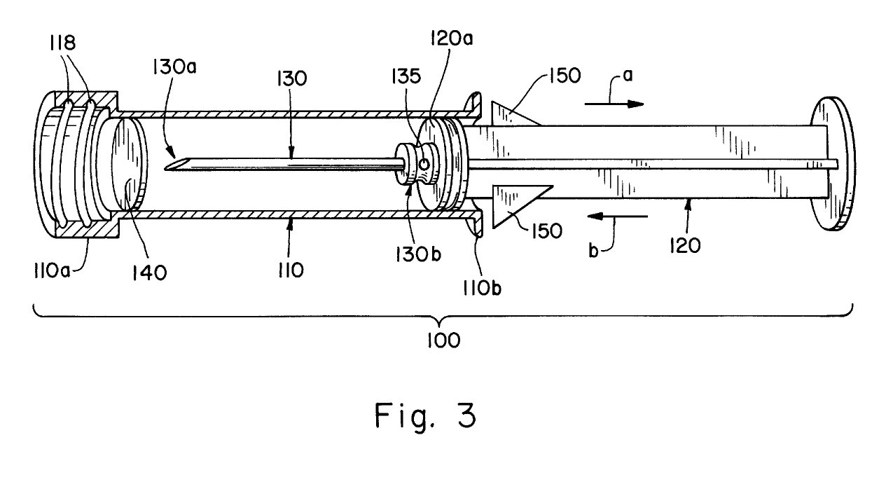

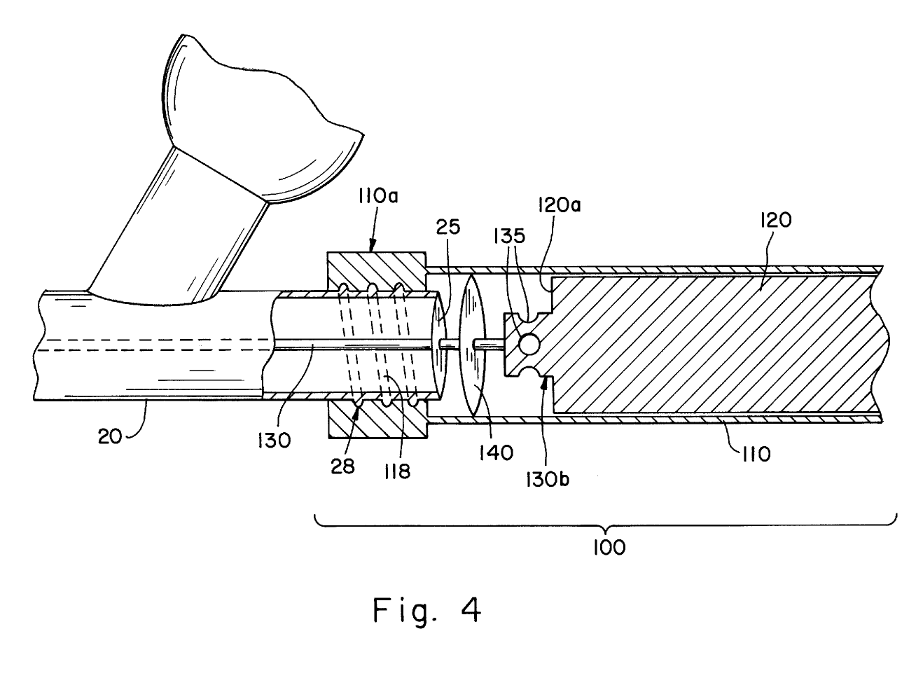

As shown in FIGS. 1 and 3, syringe assembly 100 comprises a syringe tube 110 having a distal end 110a and a plunger end 110b, a syringe plunger 120 that reciprocates back and forth within the syringe tube 110, and a hollow hypodermic needle 130 affixed to the distal end 120a of syringe plunger 120. The base 130b of the hollow needle 130 has one or more small openings 135 provided therein to allow body fluid such as blood to flow through the hollow needle 130 from its distal end 130a and into the internal volume of the syringe tube 110 when the plunger 120 is withdrawn or moved in the direction of reference arrow “a.” As shown in FIG. 4, the syringe tube 110 is provided with internal threads 118 adjacent its distal end 110a for mating with the external threads 28 of syringe arm 20 to provide a fluid-tight connection therebetween. Provided within the syringe tube 110 set back from its distal end 110a is a fluid-impermeable membrane 140.

In use, the catheter 30 is connected to a patient through the following means. The distal end 110a of syringe 110 is threadably mated with the syringe arm portion 20 of IV Y-junction 10 by means of mated threads 28 and 118 (see FIG. 4). The syringe assembly 100 is packaged with the plunger 120 fully inserted into the syringe tube 110 wherein the needle 130 is positioned forward in the direction of reference arrow “b” and the needle 130 has penetrated the fluid-impermeable membrane 140 and, secondly, the membrane 25 of the syringe arm 120, extending entirely through catheter 30, at which point its distal end 130a extends beyond the distal end 30a of catheter 30. Thus, when the syringe assembly is ready for use, the plunger 120 carrying the needle 130 is positioned as shown in FIG. 1. The needle 130 is inserted into the patient's vein by a medical professional by conventional method.

Once the needle 130 is properly located intravenously, the plunger 120 is withdrawn or moved in the direction of reference arrow “a,” thereby leaving catheter 30 positioned within the patient's vein and allowing body fluid from the patient to pass through the catheter 30 and into the interior volume of the Y-shaped junction 10. Any fluid passing through the needle 130 escapes through the opening 135 at the base of the needle into the interior volume of the syringe tube 110.

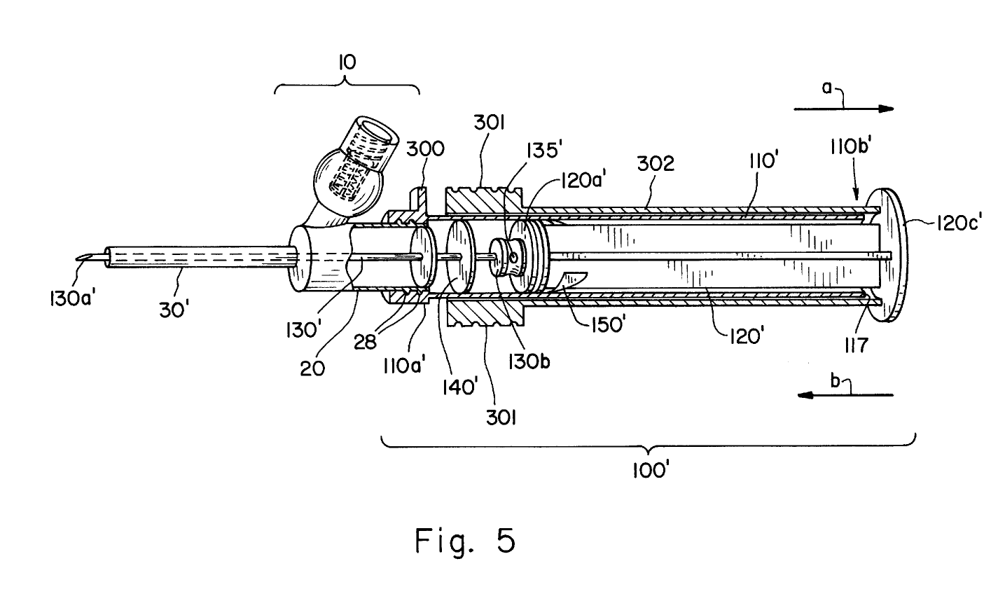

When the plunger is fully withdrawn, irreversible locking means 150 deploy to prevent the plunger 120 from re-entering the syringe tube 110 (that is, moving in the direction of reference arrow “b”). The locking means 150 do not deploy until the plunger 120 is fully retracted, generally following use by the medical professional. The locking means 150 may take a variety of forms and various suitable means are known in the art. In one embodiment, means 150 are defined by locking lugs or abutments extending radially outwardly from the long axis of the plunger 120. As noted above, during the manufacture and packaging of syringe assembly 100, plunger 120 is positioned within the syringe tube 110 whereby locking lugs 150 have been deflected in order for the syringe plunger 120 to be inserted within the tube 110. Once the plunger 120 is withdrawn to its maximum retracted position while still positioned within tube 110, and locking lugs 150 have cleared proximal end 110b of tube 110, lugs 150 rebound or flex back to their original radially outwardly extending posture as shown in FIG. 4. In this position locking lugs 150 prevent plunger 120 from being pushed back into tube 110 in the direction of reference arrow “b,” which would result in the distal end 130a of needle 130 re-penetrating membrane 140 and extending outside of the assembly 100, which would present a needle prick hazard. As shown in FIG. 5, an inner lip 117 inside the plunger end 110b of the tube 110 prevents the plunger 120 from separating from the tube 110.

Once the cannulation procedure has been completed, the needle 130 and plunger 120 have been withdrawn from the Y-junction device 10 and the syringe assembly 100 has been disconnected from device 10, the syringe assembly 100 is ready for proper disposal as medical (sharps) waste, wherein the needle 130 is captured within tube 110 to prevent inadvertent needle pricks.

While not shown in FIG. 3, the plunger 120 reciprocates within the syringe tube 110 in a fluid-tight fashion such that no liquid is able to escape from the interior volume of tube 110.

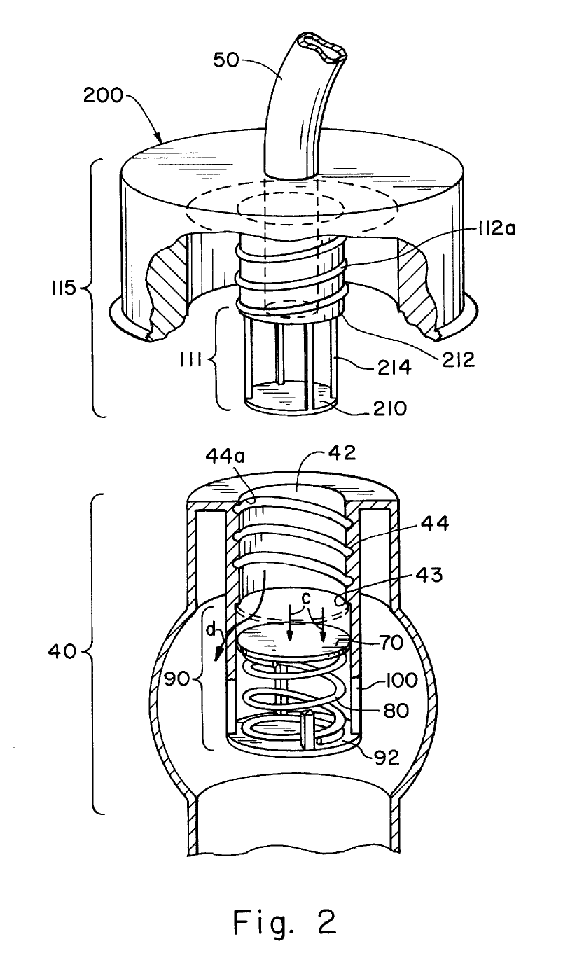

After the above steps are completed, the IV junction device 10 is at that point connected to the patient intravenously, while no IV tubing has yet been connected to the device 10. Referring now to FIGS. 1 and 2, IV tubing 50 can then be connected to the device 10 at IV connector arm 40 via a connector assembly 60, which comprises the IV connector arm 40 of device 10 and a male IV tubing connector 115 affixed to tubing 50. As shown more clearly in FIG. 2, the IV connector arm 40 is provided with an axial cavity 42 with a lower annular rim 43. Cavity 42 is further provided with internal threads 44 provided therein for threadably receiving tubing connector 115. The IV connector arm 40 also includes a liquid-impermeable seal 70 that is biased against the lower surface of rim 43 by a spring-biased device 80 held inside a cylindrical basket-style structure 90 affixed to the underside of the rim 43. The basket 90 has a solid bottom 92 but is open on its sides, and is affixed to the annular rim 43 by a series of thin vertical rails 100.

As noted, IV tubing 50 has a connector 115 that mates with the arm 40 of Y-junction device 10. Tubing connector 115 has a similar basket structure 111 that is received within axial cavity 42 of arm 40 and eventually within the basket 90 of the arm 40 in a manner that will be described further below. The IV tubing basket 111 has a solid bottom 210 that is connected to an upper portion 212 by a series of thin vertical rail members 214, but does not involve a seal and bias mechanism such as basket 90 of arm 40 does. Upper portion 212 is an annular surface affixed to the distal end of tubing 50. Connector 115 is also provided with external threads 112a disposed closely adjacent to basket 111. Canopy 200 fits over connector 115 and makes it easier for a medical worker to connect the connector 115 with the IV connector arm 40. Canopy 115 has a hollow interior and fits over the IV connector arm 40.

In use, as IV tubing 50 and connector end 115 are inserted into the Y-junction device 10 via arm 40 wherein basket 111 of connector 115 is received within the axial cavity 42 of arm 40, threads 112a of connector 115 engage and upon slight rotation form a liquid-tight seal with the female threads 44a of cavity 42 to prevent any fluid from flowing or escaping from the interior of arm 40 into the IV tubing 50. Shortly after threads 44a and 112a initially engage to form a liquid-tight seal, the lower end 210 of basket 111 first abuttingly engages the seal 70 and begins to bias it downwardly as shown by reference arrows “c,” thereby forming a path “d” allowing fluid to flow into the IV Y-junction 10 and subsequently into the patient's vein via catheter 30. As the connector end 115 is threaded onto the short arm 40, the connector end 115 increasingly biases the seal 70 of basket 90 downwardly. The threads may be continued to be rotated to lock the connector end 115, and thus the IV tubing 50, in place.

The closed system provided thereby will not allow blood or fluid to flow out of either end of the IV Y-junction 10 once it is in position within the patient's vein. The IV Y-junction 10 will have been pre-primed with saline so as to avoid air emboli when the vein is cannulated and fluid administration begun. A healthcare worker will also have primed the IV tubing 50 and connector end 115 prior to beginning the process of obtaining venous access.

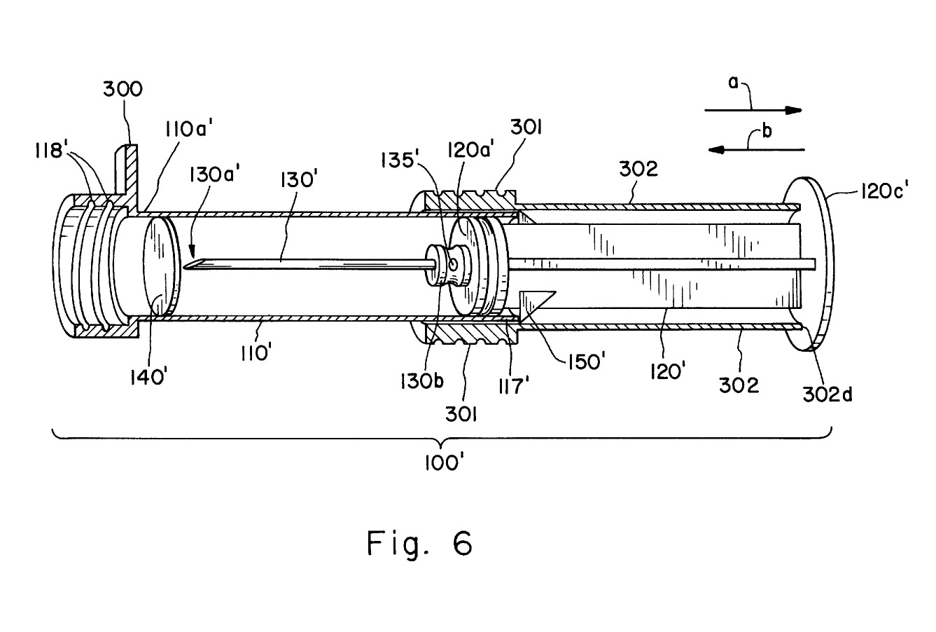

As shown in FIGS. 5-8, an alternative embodiment of the syringe assembly 100′ is adapted to fit with the present technology shown in FIGS. 1-4. This alternative embodiment allows the healthcare worker to cannulate a patient's vein without the risk of needle stick and without blatant blood exposure, and to withdraw the needle using a one-handed technique. In this alternative embodiment, the syringe assembly 100′ comprises a syringe tube 110′ having a distal end 110a′ and a plunger end 110b′, a syringe plunger 120′ that reciprocates within the syringe tube 110′, and a hollow hypodermic needle 130′ affixed to the distal end 120a′ of syringe plunger 120′. The base 130b′ of the hollow needle 130′ has one or more small openings 135′ provided therein to allow body fluid such as blood to flow through the hollow needle 130′ from the patient's vein into its distal end 130a′ and thereafter into the internal volume of the syringe tube 110′ when the plunger 120′ is withdrawn or moved in the direction of reference arrow “a.” As shown more particularly in FIGS. 5 and 6, the syringe tube 110′ is provided with internal threads 118′ adjacent its distal end 110a′ for mating with the external threads 28 of syringe arm 20 to provide a fluid-tight connection therebetween. Provided within the syringe tube 110′ set back from its distal end 110a′ is a fluid-impermeable membrane 140′.

Overlying the syringe tube 110′ is a sliding sleeve 302 that can reciprocate in an unrestricted manner. Sleeve 302 is attached to the syringe plunger end 120a′ at 302d as indicated in FIG. 6. The distal end of the sliding sleeve 302 adjacent to the distal end 110a′ of syringe tube 110′ is provided with finger grips 301, which are defined by raised, roughed, or knurled surfaces that better facilitates a healthcare worker being able to hold the sliding sleeve 302 easily as the needle 130′ is being withdrawn from the Y-junction 10 into the hollow syringe tube 110′.

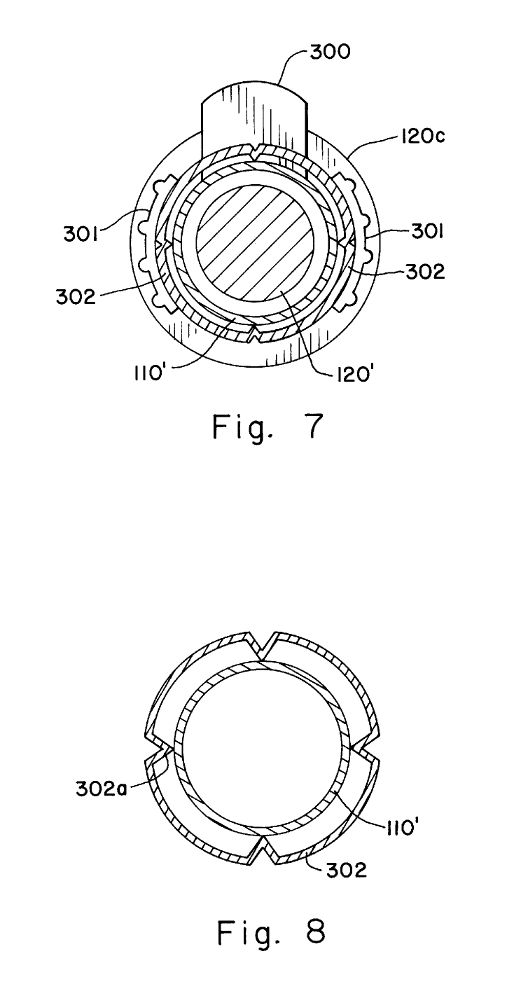

FIG. 7 is an end view of the alternative embodiment of the syringe assembly, showing the spatial relationship between the plunger 120′, the syringe tube 110′, and the sliding sleeve 302. Also shown are finger grips 301 and a push pad 300 that is attached to the distal end 110a′ of the syringe tube 110′. The finger grips 301 are located on both sides of the syringe tube 110′, in relation to the push pad 300.

FIG. 8 is an end view showing in isolation the relationship between the syringe tube 110′ and the sliding sleeve 302. To prevent or minimize the friction between the syringe tube 110′ and the sliding sleeve 302, several indentions 302a are provided in the interior of the sliding sleeve 302. Indentions 302a decrease or minimize the surface-to-surface contact between these two elements, thereby allowing the sliding sleeve 302 to slide outside the syringe tube 110′ with minimal effort.

In operation, a healthcare worker will cannulate the vein in the normal fashion. Once the vein is entered, blood will transverse the hollow needle 130′ and enter the hollow tube 110′ indicating cannulation. The healthcare worker may withdraw the needle 130′ from the catheter 30 and syringe arm portion 20 by placing one finger on the push pad 300 and one or two other fingers on the finger grips 301 of the sliding sleeve 302. The sliding sleeve 302, attached at attachment 302d to the distal end 120a′ of the plunger 120′, may then be slid in the direction of reference arrow “a” as shown in FIGS. 5-6. As the needle 130′ is withdrawn, the catheter 30 may be further advanced into the patient's vein if necessary by the healthcare worker simply advancing the push pad 300. Once the needle 130′ is fully retracted, a locking apparatus 150′ will engage and prevent the needle 130′ from re-entering the syringe tube 110′. An inner lip 117′ disposed inside the plunger end 110b′ of the tube 110′ prevents the plunger 120′ from separating from the tube 110′. The healthcare worker may then disconnect the syringe assembly 100′ from the syringe arm portion 20 by simply rotating or un-screwing the threaded connectors 28 and 118′.

Generally, in another embodiment, an intravenous catheter system consisting of a modified Y with one arm composed of a sealed system designed to mate with a connector attached to the intravenous (IV) fluid supply. Attached to the short arm end of the tubing is a hypodermic needle and syringe apparatus that extends from outside the Y through a fluid impermeable membrane and into a plastic catheter surrounding the needle outside of the Y tubing.

The syringe assembly is connected to the Y with threads that mate with corresponding threads on the Y catheter so as to provide a fluid tight seal and allow easy detachment once the needle has been withdrawn into the syringe. The syringe assembly consists of a hollow tube with the above mentioned connector on one end and houses another fluid impermeable membrane through which the needle passes. Inside this tube is a plunger device fully depressed and onto which the needle is permanently attached. The needle is hollow and has openings at its base so that blood can flow through it and into the syringe. Once IV access has been obtained and the plastic catheter is in the vein the plunger and needle are withdrawn into the hollow tube safely enclosing the needle. An attached taping device is then used to stabilize the catheter. When fully withdrawn an irreversible locking device deploys to prevent the plunger from reentering the hollow tube. This feature allows the health care worker the ability to readjust the needle and catheter if necessary to optimize the position of the catheter within the vein without the need of sticking the patient again as would be required with some automatic retractable devices.

The short arm of the Y is meant for attachment to the IV tubing and solution. It is threaded on the outside so as to mate with the IV tubing. Inside is a system composed of a valve which is held against a rim by a spring-like device held inside a basket-style container. The basket is solid on the bottom and open on the sides. The IV tubing end is manufactured with a similar basket design that mates inside the Y basket. It is open and does not require a seal and spring mechanism. The outside of the IV tubing end is also designed with threads that mate with corresponding threads of the Y connector. When the two connector ends meet and the threads begin to engage, the seal of the Y basket connector is compressed down into the basket by the mated basket of the IV connector. Once fully threaded, the IV basket will be inside the Y basket and the intravenous fluid will flow easily into the catheter and subsequently into the vein of the patient. This closed system will not allow blood to flow out of either end of the Y once it is in the vein and provides two ports, one for IV fluid and one for injection of medications if desired. Other types and combinations of ports appear in various alternative embodiments as will occur to one skilled in the art.

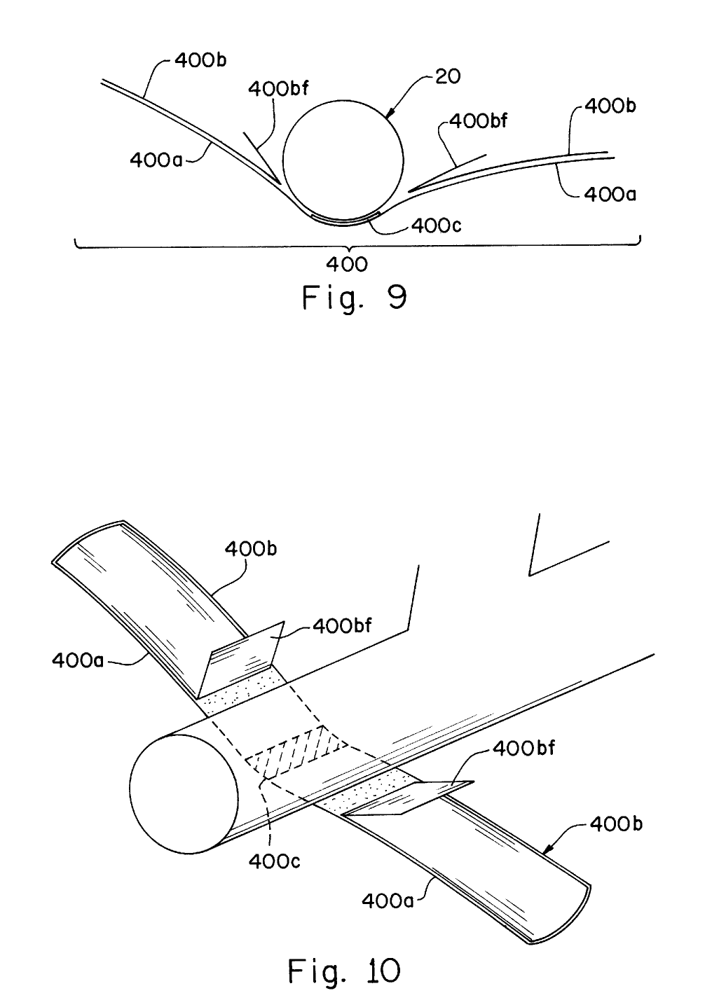

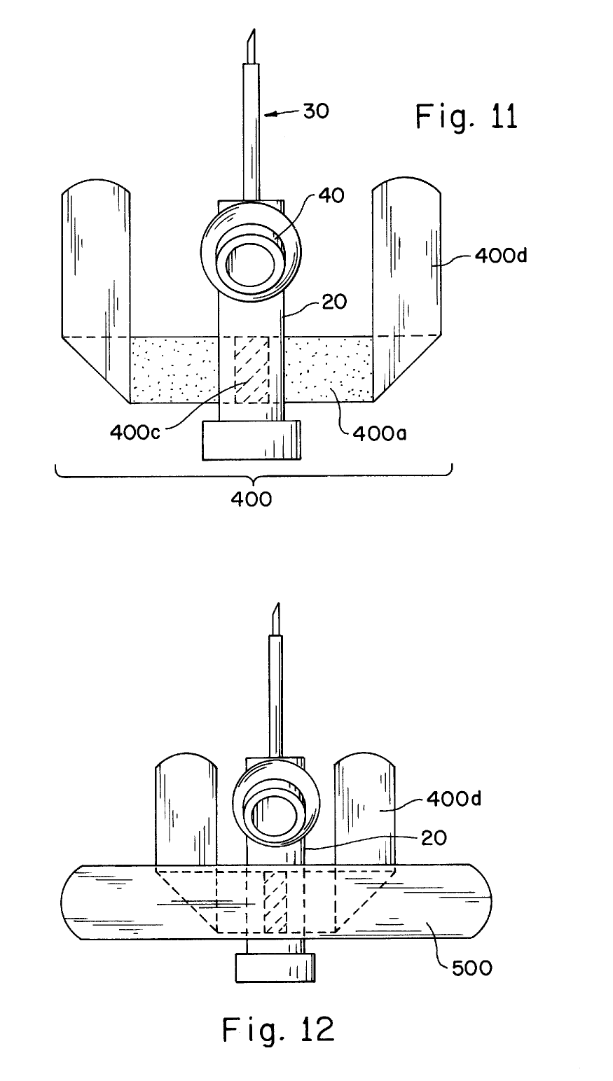

FIG. 9 is a cross section of the syringe arm portion 20 and the attached adhesive bandage 400 used for securing the syringe arm 20 and intravenous catheter 30 onto the patient. This is important because many times the intravenous access will be lost while the health care worker is attempting to tape down and secure the catheter. The customary process for accomplishing this generally requires the health care worker to tear tape from a roll and attach a section over the body of the catheter. In this embodiment, the health care worker places the tape under the catheter with the adhesive side up to make contact with the underside of the catheter, then folds each side or arm of the tape to allow the adhesive side to make contact with the skin. The final result of this step is a “U” shape of the tape with the two “arms” running parallel with the catheter in the direction of the catheter tip. Another section of tape 500 is then applied with the adhesive side down and covering the catheter and skin in a horizontal fashion as shown in FIG. 12.

FIG. 9 reveals the body of the syringe arm 20 and the adhesive bandage 400. The bandage 400 is attached to the underside of the syringe arm 20 as indicated at 400c. Extending from this attachment site are two “arms” consisting of an adhesive surface 400a facing up and a non-stick layer 400b. This layer is loosely attached to the adhesive surface 400a and can be easily peeled off by pulling on the flaps 400bf, thus exposing the adhesive surface 400a, which then can be used to secure the catheter as shown in FIG. 11. A second layer of tape can then be applied over the top of the catheter to further secure the site as shown in FIG. 12. The obverse of the adhesive bandage 400 is non-adhesive surface 400d.

FIG. 10 illustrates this adhesive apparatus in a more three dimensional view.

This adhesive bandage 400 is wrapped around the body of the syringe arm 20 and held in this position by a paper band that is easily removed (similar to that found on a roll of stamps) for packaging and during the cannulation of the vein and subsequently unwrapped for the attachment process. In this way, there is no interference from the bandage during the delicate process of inserting the catheter into the vein.

While the present technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the disclosure are desired to be protected.

What is claimed is:

1. A safety intravenous (IV) connector assembly comprising:
a Y-junction member including a syringe arm portion, a catheter portion, a fluid-impermeable membrane arranged across an inner diameter of the syringe arm portion, and an IV connector arm portion angularly offset from the syringe arm portion; and
a syringe assembly including
a tube member having a distal end and a plunger end,
a plunger situated within the tube member,
lugs, and
a hypodermic needle affixed to the distal end of the plunger, the needle having a distal end and a proximal end, and the proximal end of the needle having one or more openings formed therein;
wherein the proximal end of the needle is fixed to the plunger,
the syringe assembly is removably attached to the Y-junction member,
the plunger reciprocates within the tube member over a range of positions that allows the entire needle to be withdrawn into the tube member, and
when the entire needle is withdrawn into the tube member, the lugs move from a first position to a second position, wherein
when the lugs are in the first position, the plunger reciprocates freely, and
when the lugs are in the second position, the plunger is prevented from moving toward the distal end of the tube member.

2. The assembly of claim 1, wherein the lugs are integrally formed with the plunger.

3. The assembly of claim 1, wherein the IV connector arm portion of the Y-junction member includes a threaded end with a valve that is automatically mechanically opened when a mating IV tube connector is removably attached to the threaded end.

4. The assembly of claim 1:
further comprising a strip of adhesive tape with a removable backing strip that covers a first end and a second end of the strip of adhesive tape, but not a central portion of the adhesive tape;
wherein the central portion is attached to the outside of the Y-junction member.

5. The assembly of claim 4, wherein the central portion adheres to the outside of the Y-junction member with the same adhesive that exists on the first end and the second end of the adhesive tape.

6. The assembly of claim 4, wherein the first end and the second end of the strip of adhesive tape are wrapped around the Y-junction member and wrapped by a removable cover.
